(19) United States
(12) Patent Application Publication
(10) Pub. No.: US 2015/0343014 A1
(43) Pub. Date: Dec. 3, 2015

(54) TOPICAL ANTIMICROBIAL DERMATOLOGICAL COMPOSITION

(71) Applicant: LABORATOIRES THEA S.A.S., Clermont-Ferrand (FR)

(72) Inventors: Jean-Marie Lefevre, Amiens (FR); Jacques Peyrot, Clermont-Ferrand (FR); Jean-Claude Allart, Le Touquet (FR)

(21) Appl. No.: 14/760,781

(22) PCT Filed: Feb. 24, 2014
(86) PCT No.: PCT/FR2014/050383

§ 371 Date: Jul. 14, 2015

(30) Foreign Application Priority Data
Feb. 28, 2013 (FR) 1351766

(51) Int. Cl. A61K 38/08; A61K 9/06; A61K 31/728; A61K 9/00; A61K 45/06

(52) U.S. Cl. CPC A61K 38/08; A61K 9/0014; A61K 45/06; A61K 31/728; A61K 9/06

(57) ABSTRACT

The invention relates to a composition that may be used in dermatology.

The topical antimicrobial dermatological composition for use in human and veterinary medicine comprises in combination at least one cationic antimicrobial peptide and hyaluronic acid of medium molecular weight or a salt thereof.

Application to the treatment of cutaneous, integumental or mucosal microbial infections.

The present invention relates to a novel composition that is useful in therapeutics and which has antimicrobial properties, and more particularly to a new composition based on hyaluronic acid and on a cationic antimicrobial peptide which is useful in human and veterinary medicine, especially in dermatology and in ophthalmology for the treatment of microbial infections.

The skin is both a living anatomical area and a zone of exchange between the body and its environment, the efficacy of which has an influence on the maintenance of a good homeostatic equilibrium. The skin is itself an organ, comprising several integrated layers, starting from the surface layer, the epidermis, down to the deepest layers, the dermis and the hypodermis, in which each of these layers fulfils functions that allow the whole to react and to adapt to its environment.

The epidermis, which is composed mainly of keratinocytes, melanocytes and Langerhans cells, has a thickness that varies according to the different parts of the body, and constitutes the outer layer of the skin to protect the body against its external environment. The dermis is the thickest layer, traversed by nerve fibres and blood vessels, and is mainly composed of collagen, elastin, proteoglycans and glycosaminoglycans, mainly synthesized by the dermal fibroblasts. The collagen fibres give the skin mechanical strength and texture, elastin is responsible for its elasticity, and the glycosaminoglycans and proteoglycans play an important role in the structure and moisturization of the skin. The deepest layer of the skin forms the hypodermis, containing the adipocytes which produce lipids and form a fat layer protecting the muscles, bones and internal organs against impacts.

Changes in the structure of the epidermis, such as an increase in humidity or the existence of irritations or skin wounds of various origins, or the development of a dermatosis, promote the colonization and infection of the skin by pathogenic microorganisms. The resulting microbial proliferation has the effect of modifying the cicatrization conditions by retarding or blocking it, and by promoting the diffusion of an infection from the initial wound.

Thus, the skin is under constant attack by pathogenic microorganisms, but the cornified layer of the epidermis, on account of its pH, its relatively low water content and the presence of antibacterial peptides having bactericidal action, acts as the first line antimicrobial defence against these pathogenic microorganisms.

The majority of the bacterial colonies become biofilms, a membrane structure for microbial survival, diverting growth and cicatrization factors by creating an inflammation which prevents the antimicrobial defence process from acting efficiently. On account of the very widespread, and occasionally excessive, use of antibiotics, bacteria have developed a resistance to antibiotics which limits and even cancels out their antibacterial effects. Biofilm bacteria are resistant to the majority of antibiotics and antiseptic agents, and it is therefore necessary to develop compositions that are capable of efficiently combating pathogenic microorganisms while respecting skin homeostasis as much as possible and not promoting the development of resistant strains, which are harmful to man and his environment.

It is known that cationic antimicrobial peptides have an effect on the membrane of bacteria by electrostatic bonding between the cationic peptide and the negatively charged phospholipids found in the outer structure of the membrane of Gram-negative bacteria whereas, in the case of Gram-positive bacteria, the bonding takes place between the peptide and the components of the peptidoglycan around the plasma membrane of the bacteria. The cationic nature of the antimicrobial peptides explains their affinity for the anionic membrane of bacteria, and binding is proportionately easier the greater the charge. After binding, the peptides modify membrane permeability, forming pores which bring about the osmolysis an then the death of the bacterium.

Thus, antimicrobial peptides, in particular cationic peptides, act on the skin by protecting it against bacterial infections and inflammation.

Defensins constitute a family of natural antimicrobial peptides involved in non-specific, or innate, immunity. They are small cationic antimicrobial peptides consisting of chains of 36 to 42 amino acids comprising intramolecular disulfide bridges. In man, they have a broad spectrum of very efficient antibacterial and antifungal activities, and are divided into two groups, namely α-defensins and β-defensins. The latter are present on all epithelia, including the cutaneous epithelium and the lachrymal and buccal mucosae, and in numerous organs, and play an important role in responses to infections. β-Defensins are released following the activation of specific receptors, the Toll Like Receptors (TLRs).

Studies have shown that commensal bacterial flora and pathogenic flora use different β-defensin induction routes. Thus, the skin has molecular means for differentiating and combating bacteria.

The “Toll Like Receptors” (TLRs) are membrane receptors expressed by the family of TLR genes which have a fundamental role in the recognition of pathogenic microorganisms and the activation of innate immunity. They modulate the production of cytokines necessary for efficient immunity. TLR4 and TLR2 are active during the recognition of lipopolysaccharides (LPSs) present on Gram-negative bacteria. LPS is a pro-inflammatory bacterial endotoxin. TLR2 more particularly recognizes the peptidoglycan of Gram-positive bacteria and TLR4 recognizes the LPS of Gram-negative bacteria. In the skin, the proteins TLR2 and TLR4 are expressed on the keratinocytes and TLR2 on the sebaceous glands.

Defensins play the unusual role of elicitors, i.e. of a molecule that is capable of triggering a defence mechanism of the organism. Thus, naturally present in the skin, they are stimulated to reinforce the protection of weakened skin. In an environment that is often hostile, it is important to aid the biological functions in order to maintain the quality and condition of a sound and healthy skin. The notion of elicitor involves inciting the biological pathways present in the skin to react better in the face of external attack.

The antimicrobial properties of human β-defensin (HBD-3) are described in patent application WO 01/92309.

Hyaluronic acid is a natural disaccharide-based polymer comprising D-glucuronic acid and N-acetylglucosamine units linked via glucoside bonds. Its properties are very variable, depending on its molecular weight. Hyaluronic acids with a high molecular weight, of greater than 1000 kDa, are essentially used for promoting the moisturization of the skin by means of the hydrophilic carbohydrate network they constitute. Hyaluronic acids of low molecular weight, of less than about 50 kDa, can cross the stratum corneum barrier and stimulate the CD44 receptors responsible for the neosynthesis of hyaluronic acid in the dermis.

Hyaluronic acid, like collagen, is one of the main constituents of the extracellular matrix of the dermis and it plays an important role in cell growth and in the maintenance of moisturization. In the course of skin ageing, a decrease in its concentration in the dermis is observed. Hyaluronic acid is a glycosaminoglycan that is often used in cosmetic and dermatological compositions, generally in the form of sodium hyaluronate, especially for promoting moisturization and for stimulating cicatrization and the natural defences of the skin. It is also used in cosmetic surgery for filling wrinkles, in medical treatments for combating arthrosis, and in ophthalmology. Various crosslinked or non-crosslinked hyaluronic acid derivatives, which may show good resistance to enzymatic degradation, and which may be used in particular in cosmetic compositions, are described, for example, in patent application WO 2011/080 450. Various forms of hyaluronic acid are well known and commercially available.

It has been proposed to combine hyaluronic acid or derivatives with chondroitin sulfate, as in patent application WO 2009/073 437, or with a retinoid and an oligosaccharide, as in patent FR 2 894 827. Heavy metal salts of hyaluronic acid have also been proposed in antimicrobial compositions, as described in patent application WO 87/05517. U.S. Pat. No. 6,180,601 describes a pharmaceutical composition using a hyaluronic acid of low molecular weight, i.e. 50 to 200 kDa, forming a matrix incorporating a peptide or a protein, for example a growth hormone. Patent application WO 2006/130 974 relates to peptides preferably containing 15 to 40 amino acids, which can bind, on the one hand, to hyaluronic acid, and, on the other hand, to the bacterial capsule containing hyaluronic acid, and which may be used in the presence of a lipid to form liposomes. A pharmaceutical composition comprising a peptide derived from lactoferrin and hyaluronic acid is described in patent application WO 2010/081 800.

Studies carried out by the Applicant have demonstrated that it is possible to obtain efficient antimicrobial activity, equivalent to that of local antiseptics and antibiotics, by combining an cationic antimicrobial peptide with a hyaluronic acid of medium molecular weight, and that the antimicrobial activity of the cationic peptide is potentiated by the combination with hyaluronic acid which stimulates the production of β-defensins. The term “antimicrobial activity” means antibacterial and/or antiviral and/or antifungal activity.

The object of the present invention is to propose a novel topical composition which has antimicrobial properties, making it possible to efficiently treat cutaneous microbial infections and to reinforce the skin's natural defences, without resorting to chemical antiseptics or antibiotics.

A subject of the present invention is thus a dermatological composition comprising in combination at least one cationic antimicrobial peptide and hyaluronic acid of medium molecular weight or a salt thereof.

A subject of the present invention is also an antimicrobial topical dermatological composition comprising in combination at least one cationic antimicrobial peptide coupled to a lipid, and hyaluronic acid, or a salt thereof, with a molecular weight of between 100 kDa and 800 kDa.

Finally, a subject of the present invention is a composition comprising at least one cationic antimicrobial peptide coupled to a lipid, and hyaluronic acid of medium molecular weight or a salt thereof, in combination, for use in the treatment of cutaneous, integumental or mucosal microbial infections.

Such a composition is useful in human dermatology for treating skin wounds by virtue of its antimicrobial properties.

It is also useful in dermatology and in ophthalmology, more particularly for treating infections associated with chronic acute wounds, folliculitis, overinfections of dermatological inflammatory diseases, blepharitis, meibomitis and conjunctivitis.

According to the present invention, the cationic antimicrobial peptide is preferably a peptide comprising Less than 50 amino acids, more preferentially between 3 and 30 amino acids, and having a broad antimicrobial spectrum against Gram-positive and Gram-negative bacteria.

Use may be made, for example, of a cationic antimicrobial peptide chosen from linear peptides with a helical structure, peptides comprising one or more disulfide bridges and linear peptides rich in certain amino acids.

The cationic antimicrobial peptide of the invention comprising less than 50 and preferably less than 30 amino acids may be chosen, for example, from derivatives of magainin, protegrin, indolicidin and histatin. Use may also be made of a synthetic cationic peptide such as omiganan pentahydrochloride, which is an indolicidin analogue, iseganan hydrochloride, a synthetic protegrin, and pexiganan acetate, a magainin analogue. These peptides are coupled by covalent bonding with a lipid, and preferably with a fatty acid such as palmitic acid. More particularly, this covalent bonding is not liposomial. Use may also be made of a commercially available peptide such as Oligopeptide-10 (Grant Industries) or Shield Bact Peptide (Infinitec) which is a hexapeptide coupled to palmitic acid comprising disulfide bridges.

The antimicrobial activity of the compositions according to the invention was demonstrated via in vitro and ex vivo studies as indicated later, demonstrating a triple action consisting in neutralizing the microbial endotoxins to minimize the demands on the Toll Like Receptors, mimicking the action of the β-defensins to afford a rapid anti-infectious effect, and modulating the inflammatory reaction.

The antimicrobial peptide should preferably be amphiphilic. This amphiphilic nature is reinforced by coupling the cationic peptide with a lipid, and especially a linear or branched, saturated or unsaturated fatty acid, preferably comprising 6 to 22 carbon atoms, such as oleic acid, linoleic acid, lauric acid, sapienic acid, stearic acid or palmitic acid. The coupling of the fatty acid with the peptide preferably takes place via covalent bonding with at least one of the constituent amino acids of the peptide. The covalent bonding may be, for example, an amide bond on the N-terminal end of the peptide. The studies performed show that the amphiphilic nature thus reinforced promotes the binding of the cationic peptide to the surface of the bacteria and has, as effects:
structural disruption of the outer layer of the bacterial membrane;
accumulation of fatty acid in the form of micelles, at the membrane interface;
perforation of this double membrane by creation of ion channels.

The consequence of this triple action is bacterial lysis associated with the loss of impermeability of its membrane and with the action of the cationic peptide on the intracytoplasmic anionic targets.

Furthermore, the cationic antimicrobial peptide of the invention shows:
a broad spectrum of activity covering Gram-negative and Gram-positive bacteria and mycosal, viral and parasitic infections,
a high speed of action associated with the multiple targets attacked by the fatty acid, in the bacterium,
a very low risk of appearance of resistance when compared with that observed with conventional antibiotics.

The studies carried out also showed that the antibacterial cationic peptide used in the invention is capable of neutralizing the response of host cells to bacterial endotoxins, irrespective of the type, depending on the bacteria (Gram-negative or Gram-positive) that they produce. It thus modulates the innate immune response by limiting the activity of the TLRs and the production of TNF-α, which attenuates the inflammatory cascade and the resulting cytokine storm (IL-6, IL-1b, IL-1a).

It also has other properties in connection with innate immunity by the chemotactic action on monocytes and polynuclear neutrophils, by the release of histamine by the monocytes, and by inhibiting proteases limiting tissue destruction and simulating tissue repair.

The studies carried out have shown that this innate immune defence mechanism in the presence of a bacterial attack can be optimized by combining the cationic antimicrobial peptide, preferably linked to an amino acid, with hyaluronic acid of medium molecular weight. The immediate action on the microorganisms is supplemented by neutralization of the endotoxins they secrete.

As a result, the release of pro-inflammatory cytokines is reduced and the intrinsic action of hyaluronic acid can then be fully expressed by accelerated binding to the Toll-Like Receptors and increased secretion of β-defensins, thus creating a second line of defence against microorganisms. In parallel, repair mechanisms are stimulated to rapidly restore the barrier effect of the epidermis.

According to the present description, the term “hyaluronic acid” means hyaluronic acid in free form or in the form of an alkali metal or alkaline-earth metal salt thereof, for example sodium, potassium, calcium or magnesium hyaluronate, of medium molecular weight, i.e. preferably between 100 and 800 kDa and more preferentially between 200 and 600 kDa.

The hyaluronic acid that may be used in the invention is commercially available in various forms that are adapted to their intended uses. It may be produced industrially in large amounts by extraction from animal tissues such as cockscombs, or by bacterial fermentation, or alternatively via a biotechnological process from plant substances, for example wheat.

Use may be made, for example, of hydrolysed hyaluronic acid or of the sodium salt of hyaluronic acid, with a molecular weight of between 200 and 600 kDa, such as the products commercially available under the brand name PrimalHyal 300 (Soliance) with a molecular weight close to 300 kDa or PrimalHyal 450 with a molecular weight in the region of 450 kDa.

The compositions according to the present invention may comprise an effective amount of each of the above active agents, for example between 0.05% and 2% by weight of hyaluronic acid, and between 0.001% and 1% by weight of cationic antimicrobial peptide, relative to the total weight of the composition.

The compositions according to the invention may also comprise one or more additional active agents that advantageously reinforce or supplement the activity of the combination of hyaluronic acid and antimicrobial peptide, and which are compatible, i.e. not liable to react with each other or to mask or limit their respective effects.

For example, they may contain chitosan, or chitin, which, by combining with hyaluronic acid, has a protective crosslinked network effect which promotes the endogenous production of keratinocyte growth factors, inducing fibroblast activity, increasing collagen production and accelerating the differentiation of fibroblasts and myofibroblasts. They may also contain a sphingoid base, such as sphingamine and phytosphingosine, which optimizes the antibacterial effect and reinforces the anti-inflammatory effect of the peptide.

Various secondary active agents may be advantageously added to the composition and chosen, for example, from a cicatrizing agent, an anti-inflammatory agent, an anti-infectious agent and a vitamin such as vitamin A or E. The cicatrizing agent may be, for example, a zinc salt or a locust bean extract rich in oligogalactomannans. The anti-inflammatory agent may be a polysaccharide such as Rhamnosoft® or Teflose® (Solabia) which inhibits cellular adhesion and limits inflammatory reactions, or an extract of Boswellla serrata (Soothex®) which acts via enzymatic inhibition of leukotriene synthesis. The additional anti-infectious agent may be chosen from a silanol and an antimicrobial peptide formation activator such as methyl caproyl tyrosinate (Defensamide®).

The compositions according to the present invention may be in any galenical form that is common for topical application, especially for external topical application. They may be, for example, in the form of aqueous or aqueous-alcoholic solutions, micellar solutions, solutions for spraying, shampoos, dispersions, serums, wipes, patches, meshes or dressings with controlled release, gels (aqueous, anhydrous or lipophilic), oleogels (lipid gels), ointments, suspensions, ionic or nonionic vesicular dispersions, and liquid or semi-liquid (for example a milk), solid or semi-solid emulsions. The emulsions may be of the oil-in-water (O/W) or water-in-oil (W/O) type, for example gels or creams.

The excipients and supports that may be used for the preparation of the compositions in accordance with the present invention are those commonly used in preparations for dermatological use, and are chosen as a function of the selected form of administration. Examples that may be mentioned include emulsifiers, thickeners, gelling agents, softeners, preserving agents, solubilizers, suspension agents, and also washing bases and fragrances.

The emulsifier may be chosen, for example, from high molecular weight carboxyvinyl polymers, polysorbates such as Polysorbate 20® or Tween 60®, sorbitan esters and more particularly a sorbitan stearate, palmitate or laurate such as Arlacel®. Emulsifiers that may also be used include a stearic or palmitic acid derivative, for example polyethylene glycol stearate, glyceryl stearate, PEG 100 Stearate® (for example Arlacel 165®), a steareth or a ceteareth, a fatty alcohol such as a stearyl, caprylyl or ceteary alcohol, for example Montanov 68®, or an emulsifiable silicone.

The gelling agents and thickeners are incorporated into the composition to improve its fluidity. They may be chosen, for example, from polyacrylamides of the Carbopol type, acrylate/acrylic acid copolymers such as Aculyn®, crosslinked acrylates such as a Carbopol Ultrez®, cellulose derivatives such as hydroxypropyl cellulose, or natural gums such as xanthan gum and gum tragacanth.

The moisturizers and softeners used in the composition may be chosen, for example, from propylene glycol, glycerol, butylene glycol and shea butter, and also fatty alcohols. A suitable suspension agent is, for example, a clay such as bentonite or smectite.

An agent that facilitates penetration into the epidermis, such as cosmoperine, may also advantageously be added.

The compositions in accordance with the present invention are prepared via the usual techniques, as a function of the chosen form of administration, the desired amounts of hyaluronic acid, or of salts thereof, being mixed with the antimicrobial peptide, and optionally chitin or chitosan, and the supports and excipients, in a physiologically acceptable medium.

For the purposes of the present invention, the term “physiologically acceptable” means supports and excipients of a type commonly used in dermatological compositions in human medicine, which are neutral with respect to the active principles used, which have no toxic effect and which give rise to no harmful side effects on the skin.

For example, in the case of a cream, the process may be performed by dispersing a fatty phase in an aqueous phase to obtain an oil-in-water emulsion, or, conversely, to prepare a water-in-oil emulsion, the active principles being in one or the other phase.

The composition of the invention, for example in the form of a cream, is preferably applied two to three times per day to the area of skin requiring the treatment, for a period of time which may range from a few days to four weeks depending on the severity of the complaint.

The examples that follow illustrate the invention in greater detail without limiting its scope. In all the composition examples that follow, the parts are expressed on a weight basis, unless otherwise mentioned.

### EXAMPLE 1

An aqueous get is prepared by mixing at room temperature the components in the order indicated below.

| | |
|---|---|
| Demineralized water | 78.60 |
| Carbopol Ultrez 20 | 0.80 |
| Sodium hydroxide (32% aqueous solution) | 0.50 |
| Glycerol | 2.00 |
| Shield Bact Peptide ® (0.1% solution) | 5.00 |
| Demineralized water | 10.00 |
| Hyaluronic acid (PrimalHyal 450 ®) | 0.10 |
| Demineralized water | 2.00 |
| Pentylene glycol | 1.00 |

An aqueous gel thus composed may be used typically two to three times per day for several consecutive days depending on the severity of the treated complaint.

### EXAMPLE 2

A lotion having the composition below is prepared via a standard technique.

| | |
|---|---|
| Demineralized water | 82.65 |
| Carbapol Ultrez 20 | 0.45 |
| Sodium hydroxide (32% aqueous solution) | 0.30 |
| Demineralized water | 7.00 |
| Hyaluronic acid (PrimalHyal 450 ®) | 0.10 |
| Glycerol | 2.00 |
| Teflose ® (Solabia) | 2.00 |
| PEG-7 olive oil ester | 2.00 |
| Polysorbate 20 | 0.50 |
| Shield Bact Peptide ® (0.1% solution) | 3.00 |

This lotion may be used once to twice per day for several days for the treatment of ophthalmic complaints such as blepharitis.

### EXAMPLE 3

An antimicrobial cream is prepared via the usual techniques by successively mixing the phases containing the components indicated below, phases A and B being mixed while warm (65-70° C.), phase C then being added at 50° C., with careful mixing, followed by phase D.

Phase A

| | |
|---|---|
| Nonionic emulsifier (Montanov 68) | 4.00 |
| Myristyl alcohol | 2.00 |
| Stearyl alcohol | 0.50 |
| Cetiol RLF ® | 3.00 |
| Glyceryl stearate | 3.00 |
| Oleic sunflower oil | 4.00 |

Phase B

| | |
|---|---|
| Demineralized water | 67.90 |
| Carbopol Ultrez 21 ® | 0.45 |
| Propanediol | 2.00 |

Phase C

| | |
|---|---|
| Glycerol | 2.00 |
| Shield Bact Peptide ® (0.1% solution) | 6.00 |

Phase D

| | |
|---|---|
| Sodium hydroxide (32% aqueous solution) | 0.05 |
| Demineralized water | 5.00 |
| Hyaluronic acid (PrimalHyal 300 ®) | 0.10 |

The pH of this composition is adjusted to 6.5 by addition of 32% sodium hydroxide.

This cream may be used by application to the areas of skin to be treated, once to twice per day for a period of time that is determined by the physician, and which may range from one to three weeks depending on the nature and severity of the treated complaint.

### EXAMPLE 4

The effect of the choice of hyaluronic acid on the production of β-defensins and keratinocyte proliferation was measured as follows.

| | | Keratinocyte |
|---|---|---|
| | Beta-defensins | proliferation |
| hmw HA | 0 | NS |
| mmw HA 1 | ++++ | S |
| lmw HA 2 | + | NS |
| vlmw HA 3 | (+) | NS |

In the above table, the “+” sign represents the efficacy, from very poor (+) to very strong (++++). NS means “not significant”.

The hyaluronic acid (HA) used is of high molecular weight (hmw), medium molecular weight according to the invention (mmw—about 450 kDa), low molecular weight (lmw) or very low molecular weight (vtmw).

Production of β-Defensins:

Keratinocyte cultures at confluence were treated for 18 hours with a 0.2% mmw HA, or a positive control (LPS from Escherichia coli 5 μg/ml) or with:
on the one hand, a high molecular weight hyaluronic acid (hmw HA)
on the other hand, low molecular weight hyaluronic acids (HA of 20 kDa and HA of 10 kDa).

The release of HBD2 was evaluated in the supernatant by means of an ELISA kit. The values presented are averages of the results.

| | Control | LPS | mmw HA | lmw HA | vlmw HA |
|---|---|---|---|---|---|
| pg/ml | 11 | 43 | 105 | 20 | 18 |

Keratinocyte Proliferation:

On D0, a wound is made on a keratinocyte monolayer (HaCaT) at confluence. The cells are then treated at 37° C. in 2.5% FCS (foetal calf serum) without HA (reference condition) or with HAs of increasing molecular weights (HA 50, HA 300, HA 1000).

The width between the two edges of the wound is measured at 3 different points on the same photograph for each treatment at D0 and D1. Each experiment is performed 5 times independently. The means are processed statistically (one-factor analysis of variance followed by a Dunnett test (SigmaStat).

Only the mmw HA (HA 300) makes it possible to accelerate the cutaneous repair process via keratinocyte proliferation (HaCat).

Stimulation of Bacterial Defences:

3 solutions are prepared:

Solution A: 0.10% mmw HA

Solution B: 0.05% hexapeptide

Solution C: 0.05% hexapeptide+0.10% mmw HA

The hexapeptide used is Shield Bact Peptide® which is commercially available (Infinitec). The hyaluronic acid (mmw HA) has an average molecular weight of 300 kDa.

The protein from human skin biopsies after treatment (solutions A, B, C or the medium alone) are extracted with gentle stirring for 2 hours in a solution containing 5% acetic acid and protease inhibitors (0.02 mM PMSF, 2 ng/ml pepstatin and 2 ng/ml leupeptin).

The soluble protein of the supernatant is dried under vacuum and homogenized in a 0.01% acetic acid solution.

10⁴ bacteria/ml of E. coli (ATCC 4157) are incubated in logarithmic growth medium in a phosphate buffer (pH 7.4) and a final volume of 100 μl.

This E. coli suspension is mixed with 30 μg of protein extract and with 10 μl of 0.01% acetic acid solution containing the protease inhibitors (negative control) and the mixtures are incubated for 120 minutes at 37° C.

At the end of the incubation, 10 μl are taken and 1/10 and 1/100 dilutions are seeded in triplicate in Petri dishes containing casein-soya broth and incubated overnight at 37° C.

After seeding in a Petri dish and incubation overnight at 37° C., the CFU number is determined for each sample.

The table of results below shows that the combination of antimicrobial hexapeptide coupled to palmitic acid with medium molecular weight hyaluronic acid according to the invention (solution C) proves to be the most effective, the percentage inhibition of CFU formation being 100%.

| | Control | Sol. A | Sol. B | Sol. C |
|---|---|---|---|---|
| % inhibition | 40 | 55 | 77 | 100 |

### EXAMPLE 5

Two clinical studies were performed on dogs of different breeds and one clinical study was performed on patients, in human dermatology.

First Study on Dogs:

Five dogs of different breeds from 3 to 10 years old presenting an infectious blepharo-conjunctivitis in both eyes were treated with solutions prepared as follows:
solution A: a micellar solution containing 0.10% of mmw HA and 0.03% of hexapeptide, according to the invention, was applied to the right eye
solution B: a solution containing 0.03% of hexapeptide was applied to the left eye.

The average molecular weight of the hyaluronic acid of the composition is 450 kDa and the hexapeptide is Shield Bact Peptide (Infinitec).

The clinical evolution was monitored at 4, 8, 12, 24 and 48 hours. The results are reported in the table below.

| Evolution | Dog | Right eye | Left eye |
|---|---|---|---|
| 4 hours | 1 | + | + |
| 8 hours | 2 | ++ | +(+) |
| 12 hours | 3 | +++ | ++ |
| 24 hours | 4 | +++ | ++ |
| 48 hours | 5 | ++++ | +++ |

The results show a faster improvement in the infectious symptomology with solution A according to the invention which is expressed from the 8^{th} hour and is confirmed at 12 and 24 hours.

Second Study on Dogs:

Case 1: Airedale terrier (10 years old) presenting a dry kerato-conjuntivitis for several years and treated unsuccessfully with cyclosporine. The eye is dry, with overinfected conjunctivitis and reduction of the palpebral fissure. The application of lotion A above once a day for 8 days brings about a spectacular improvement (clean, open eye, with disappearance of the pain).

Case 2: Maltese Bichon (15 years old) presenting severe corneal ulceration following an anaesthesia of long duration, without improvement with the usual treatments. The eye remains dry, the ulceration is deep and the pain is acute. The use of lotion A according to the invention twice a day for 8 days brings about cicatrisation and disappearance of the pain.

Case 3: West Boxer (7 years old) presenting corneal ulceration with 8 mm granulation tissue. Healing is obtained by using lotion A twice a day for 8 days.

Case 4: Tzu Bans presenting dry kerato-conjunctivitis with corneal ulceration and pain. A customary treatment for 3 weeks remains ineffective. The use of lotion A according to the invention twice a day for 8 days brings about a substantial attenuation of the pain without any marked modification of the ulceration. Changing to cyclosporine brings about a clinical improvement of the ulceration in 8 days.

Case 5: Kitten presenting severe coryza complicated by a perforation of the ocular globe. An antibiotic therapy does not afford any improvement. Failure of a therapeutic palpebral occlusion. Lotion A according to the invention is then used 3 times a day and results in a rapid improvement.

Study in Man:

Atopic dermatitis is characterized by a particular sensitivity to Staphylococcus aureus. Its role is underlined during eczema outbursts and control of proliferation on the skin's surface is essential for improving the symptomology.

Four adult patients, 2 men and 2 women, from 18 to 31 years old, presenting an overinfected eczema outburst located in the major anatomical folds (crook of the arm, popliteal spaces) were treated with two fluid emulsions:
emulsion A comprising the hexapeptide alone at 0.05%, applied to the right side;
emulsion B comprising the hexapeptide (0.05%) and mmw HA (0.20%) applied to the left side.

The average molecular weight of the hyaluronic acid is 300 kDa. The hexapeptide is Shield Bact Peptide (Infinitec).

The pruritus, seeping and overinfection criteria were noted from 0 to 4 so as to define an evolution scoring.

| | T0 | | T12 | | T24 | | T48 | | |
|---|---|---|---|---|---|---|---|---|---|
| SYMPTOMS | A | B | A | B | A | B | A | B | RESULTS |
| PRURITUS | 4 | 4 | 3 | 2 | 2 | 1 | 2 | 1 | B > A |
| SEEPING | 4 | 4 | 2 | 1 | 1 | 0 | (1) | 0 | B > A |
| OVERINFECTION | 4 | 4 | 2 | 1 | 1 | 0 | 0 | 0 | B > A |

These results show that both creams are effective, but that cream B has faster action on the symptomology, which is manifested from the 12^{th} hour.

The overinfection is controlled at 24 hours.

The overall improvement is more pronounced with cream B according to the invention.

1. Topical antimicrobial dermatological composition for use in human and veterinary medicine, comprising:
in combination at least one cationic antimicrobial peptide coupled to a lipid, and
hyaluronic acid with a molecular weight of between 100 kDa and 800 kDa, or a salt thereof.

2. Dermatological composition according to claim 1, wherein the hyaluronic acid has a molecular weight of between 200 kDa and 600 kDa.

3. Composition according to claim 1, wherein the hyaluronic acid salt is chosen from alkali metal and alkaline-earth metal salts.

4. Composition according to claim 3, wherein the hyaluronic acid salt is sodium, potassium, calcium or magnesium hyaluronate.

5. Composition according to claim 1, wherein the antimicrobial peptide is coupled to a lipid via covalent bonding.

6. Composition according to claim 5, wherein the antimicrobial peptide is coupled via covalent bonding to a fatty acid containing 6 to 22 carbon atoms.

7. Composition according to claim 6, wherein the fatty acid is chosen from oleic acid, linoleic acid, lauric acid, sapienic acid, stearic acid and palmitic acid.

8. Composition according to claim 1, wherein the antimicrobial peptide is chosen from linear peptides of helical structure and peptides comprising one or more disulfide bridges.

9. Composition according to claim 8, wherein the antimicrobial peptide is a hexapeptide coupled to palmitic acid comprising disulfide bridges.

10. Composition according to claim 1, wherein said composition also comprises chitin or chitosan.

11. Composition according to claim 1, wherein said composition comprises between 0.005% and 2% by weight of hyaluronic acid, and between 0.001% and 1% by weight of cationic antimicrobial peptide, relative to the total weight of the composition.

12. Composition according to claim 1, wherein said composition is in a form for external topical application.

13. Composition according to claim 1 wherein said composition also comprises one or more secondary active agents chosen from a cicatrizing agent, an anti-inflammatory agent, an anti-infectious agent and a vitamin such as vitamin A or vitamin E.
